(19) United States
(12) Patent Application Publication
(10) Pub. No.: US 2016/0045155 A1
(43) Pub. Date: Feb. 18, 2016

(54) METHOD OF ASSESSING RESPIRATORY EFFORT

(71) Applicant: DYMEDIX CORPORATION, Shoreview, MN (US)

(72) Inventors: James P. Moore, Bloomington, MN (US); Todd M. Eiken, Lindstrom, MN (US)

(21) Appl. No.: 14/459,091

(22) Filed: Aug. 13, 2014

(51) Int. Cl. A61B 5/00; A61B 5/08

(52) U.S. Cl. CPC A61B 5/4818; A61B 5/0803

(57) ABSTRACT

A method of measuring a patient's respiratory effort that does away with the need for body encircling belts comprises a PVDF sensor sandwiched between a covering layer of a single sided adhesive tape strip and a bottom layer of a double sided adhesive tape strip arranged such that the sensor may be adhesively affixed to a person undergoing a sleep study and where electrical leads cooperating with the PVDF sensor are adapted to be connected to a physiological data collection device such as a PSG machine or a HST monitor.

### CROSS REFERENCE TO RELATED APPLICATIONS

None

### STATEMENT RE FEDERALLY SPONSORED RESEARCH

None

### BACKGROUND OF THE INVENTION

I. Field of the Invention

This invention relates generally to methods of conducting sleep studies on patients to assess apnea and more particularly to a method for assessing respiratory effort that obviates the need for conventional RIP Belts.

II. Discussion of the Prior Art

In conducting sleep studies on patients suspected of episodes of sleep apnea, it is customary to apply a number of sensors to the patient for detecting such things as respiratory air flow, snoring, rapid eye movement, leg twitches, heart rate, and respiratory effort. Conventionally, respiratory inductance plesmography belts (RIP belts) are used to monitor respiratory effort. The RIP belts incorporate a body encircling band incorporating a somewhat sinusoidally bent wire. A high frequency signal is applied and as the patient breathes, chest or abdominal movement causes a measurable modulation of the applied signal that is delivered over electrical leads to a polysomnograph (PSG) machine, along with outputs from the other sensors where by abnormal sleep events can be assessed. Such RIP Belts have been found to suffer from a number of problems, not the least of which is the breakage of the undulating inductance wire that necessitates a redo of the sleep study. Additionally, RIP belts have the potential to reverse polarity of the output waveform should the patient move so as to fold the elastic RIP belt a certain way. Cleaning the reusable belt can also be problematic as water will affect and corrode the embedded wire over time.

Dymedix Corporation of Shoreview, Minn. introduced to the market a respiratory effort belt to replace a RIP Belt. Instead of a variable inductance element, it incorporated a piezoelectric element that generates a measurable signal when subjected to changes in force on the element as the patient's chest and/or abdomen changes in circumference due to breathing. The piezoelectric element in the Dymedix belt is a strip of polyvinylidene fluoride (PVDF) polymer disposed on a predetermined surface of the body-encircling belt. The film is metalized on its two major surfaces and electrical leads are conductively attached to the metallization to convey signals to a PSG machine. Further information may be obtained from published U.S. Patent Application No. 2008/0275356A1. The present invention provides a method of assessing respiratory effort of a patient that eliminates the need for a belt configuration in conducting a sleep study.

### SUMMARY OF THE INVENTION

A method of detecting, identifying, monitoring and assessing a sleeping patient's respiratory effort involves the steps of providing a sensor comprising a flat strip of PVDF polymer having a metallized layer on its opposed major surfaces to which are conductively attached a pair of insulated electrical leads. The metallized strip and the distal end portions of the leads are sandwiched between a layer of double sided adhesive tape and a layer of single sided adhesive tape. This construction allows the sensor to be adhesively affixed to the patient's skin on the chest and/or abdominal area. The proximal ends of the leads are then connected to a physiological data collection device, such as a PSG machine or a home sleep test (HST) monitor or other suitable physiological data collection device whereby electrical signals due to changes in thoracic or abdominal circumference resulting from breathing are generated and analyzed for purposes of screening or diagnosing sleep disordered breathing abnormalities.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which:

FIG. 1 is an exploded view of a sensor used in carrying out the method of the present invention;

FIG. 2 is a schematic drawing of a person's torso with the sensor of FIG. 1 attached; and

FIG. 3 is a process flow diagram of the steps for carrying out the method of the present invention.

### DESCRIPTION OF THE PREFERRED EMBODIMENT

Referring to FIG. 1, there is indicated generally by Numeral 10 a piezoelectric sensor used in carrying out the method of the present invention. It is seen to comprise a strip of a PVDF polymer 12 on whose major surfaces are deposited metallization layers 14 and 16. The PVDF strip is preferably rectangular and may be about 6 centimeters long and 2 centimeters wide. Affixed to the metallization layers by a suitable conductive adhesive are conductive tabs 18 and 20 to which insulated electrical leads 22 and 24 are conductively joined. A strip of single sided adhesive tape 26 whose length and width dimensions exceed those of the PVDF polymer strip 12 is laminated with a layer 28 of a double sided adhesive tape strip. Without limitation, the adhesive strips may be of a length in a range between 6 and 24 centimeters and may be of a width in a range between 2 and 4 centimeters. The PVDF strip with the metallization layers 14 and 16 and distal end portions of the electrical leads 22 and 24 are effectively sandwiched between the tape layers 26 and 28. A strip of silicone coated release paper 30 protects the integrity of the adhesive on the double sided tape strip 28 prior to application of the sensor 10 to a patient in the course of a sleep study.

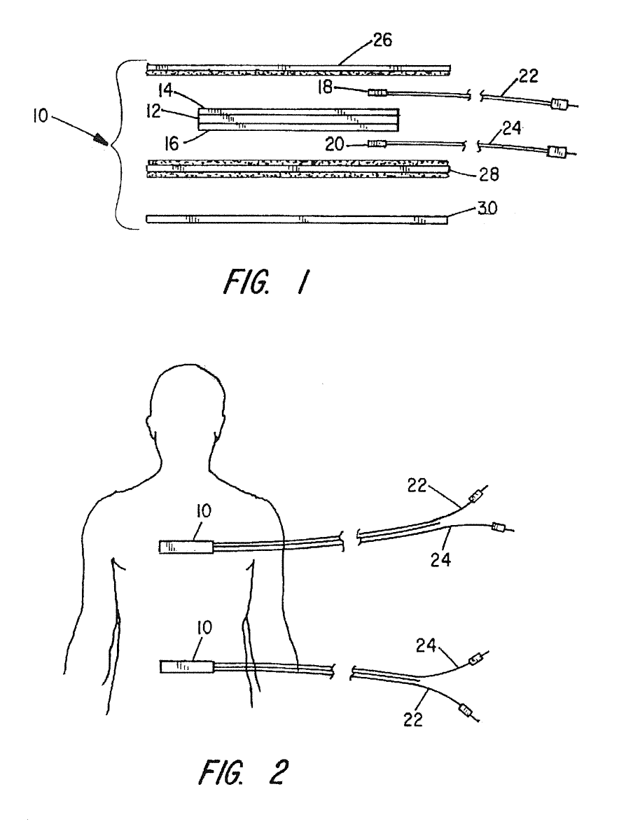

Referring to FIG. 2, two such sensors 10 may be placed on a person, one being appended to the chest area and the other to the abdominal area.

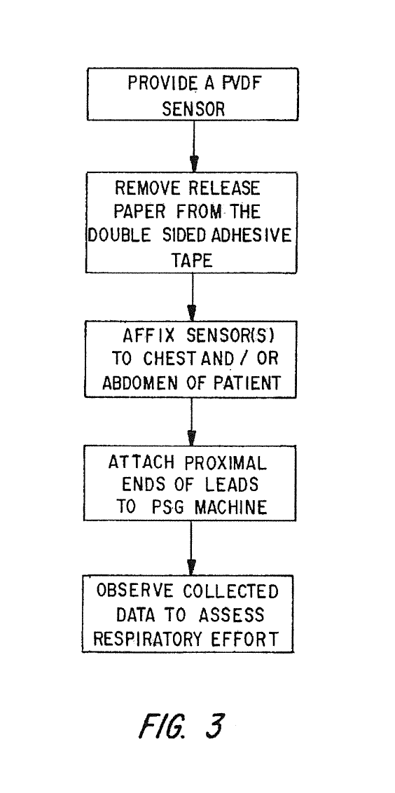

Referring to the flow diagram of FIG. 3, in use, the release paper layer 30 is first removed from the sensor module 10 exposing the adhesive on the lower surface of the double sided adhesive tape strip 28 and thus allowing the sensor to be affixed to the patient's skin either one of the chest area or the abdominal area as shown in FIG. 2. A second such sensor 10 may then be adhesively affixed to the other of the chest or abdominal areas. Once the sensors 10 are affixed to the patient, their associated leads 22 and 24 may be plugged into a suitable physiological data collection device, e.g., a PSG machine or a HST monitor as the case may be.

As the patient breathes, the circumference of the chest and abdominal areas will expand during inhalation and will fall during exhalation. The resulting stretching and recovery of the PVDF polymer layer 12 causes a voltage to be applied to the leads 22 and 24 creating a signal proportional to the force exerted on the transducer. The signal is recorded and/or displayed for analysis by a medical professional who can then assess the patient's respiratory effort.

It will be immediately recognized that the need for a body encircling belt is obviated. Upon completion of the sleep study, the sensor may be readily removed from the patient and disposed of.

It is apparent that there has been provided in accordance with this invention a disposable sensing device for sensing respiratory effort. While the invention has been described in combination with a specific embodiment thereof, it should be evident that many modifications and variations will become apparent to those skilled in the art from what is described herein. Accordingly, it is intended to embrace all such alternatives, modification and variations as fall within the spirit and broad scope of the appended claims.

What is claimed is:

1. A method of detecting, identifying, monitoring and assessing respiratory effort of a person comprising the steps of:
a. providing a sensor including a flat strip of a PVFD polymer of a predetermined length dimension having a layer of metallization on opposed major surfaces thereof and with first and second elongated, conductive, insulated leads individually in electrical contact at a first end with the layers of metallization, said metallized flat strip being sandwiched between a layer of single sided adhesive tape and a layer of double sided adhesive tape;
b. adhesively affixing the sensor to the skin of the person proximate either the chest or the abdomen; and
c. connecting a second end of the first and second leads to a physiological data collection device whereby electrical signals due to changes in thoracic or abdominal circumference resulting from breathing are generated and analyzed for either screening or diagnosing sleep disordered breathing abnormalities.

2. The method of claim 1 wherein one side of the double-sided adhesive tape is provided with a covering layer of a silicone release paper and further comprising a step of removing the release paper to expose the adhesive on the one side prior to affixing the sensor on the person.

3. The method of claim 1 wherein the physiological data collection device is a PSG machine.

4. The method of claim 1 wherein the physiological data collection device comprises a home sleep test recorder.

5. The method of claim 1 wherein said predetermined length dimension is in a range from 6 centimeters to 24 centimeters.
